(19) United States
(12) Patent Application Publication
(10) Pub. No.: US 2011/0071169 A1
(43) Pub. Date: Mar. 24, 2011

(54) Preparation of polymorphic form of lapatinib ditosylate

(75) Inventors: Hui Min He Huang, Shrewsbury, MA (US); Hu Yang, Andover, MA (US)

(73) Assignee: MAI DE LTD

(21) Appl. No.: 12/806,466

(22) Filed: Aug. 14, 2010

Related U.S. Application Data
(60) Provisional application No. 61/275,120, filed on Aug. 26, 2009.

(51) Int. Cl. A61K 31/517; C07D 405/10; A61P 35/00

(52) U.S. Cl. 514/266.24; 544/293

(57) ABSTRACT

The present invention is directed to two novel polymorph form (Form A and Form B) of lapatinib ditosylate, wherein Form A is the hydrate ditosylate salt of lapatinib, and Form B is anhydrate ditosylate salt of lapatinib. The present invention is further directed to amorphous form of lapatinib ditosylate and its solid dispersion. The present invention further provides processes for the preparation of Form A, Form B, Amorphous form and solid dispersion of lapatinib ditosyalte, and a pharmaceutical composition comprising the said forms. Form A and Form B were characterized by X-RPD, DSC, TGA and FT-IR, and can be prepared from recrystallizing lapatinib ditosylate in a mixture of tetrahydrofuran (THF) and water.

This application claims the benefit of priority to U.S. Provisional Patent Application Ser. No. 61/275, 120 filed on Aug. 26, 2009, the entire disclosure of which is hereby incorporated by reference.

### FIELD OF THE INVENTION

The present invention is directed to novel polymorphic forms of crystalline lapatinib ditosylate, novel amorphous lapatinib ditosylate, to processes for preparing said polymorphic form, to pharmaceutical compositions comprising the same, and to methods of treatment using the same.

### BACKGROUND OF THE INVENTION

The lapatinib ditosylate salt, its chemical name is N-[3-chloro-4-[(3-fluorophenyl) methoxy]phenyl]-6-[5-[[[2-(methylsulfonyl)ethyl]amino]methyl]-2-furanyl]-4-Quinazolinamine, bis(4-methylbenzenesulfonate). The chemical structure of lapatinib ditosylate salt is shown as follows:

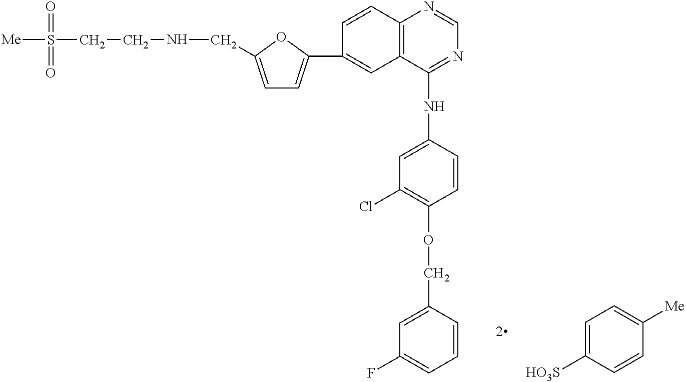

Lapatinib and its acid addition salts are disclosed in U.S. Pat. No. 6,727,256 (or WO 99/35146). Lapatinib and its salts are inhibitors of protein tyrosine kinase (PTK), such as erbB family PTKs, and useful in the treatment for disorders characterized by aberrant erbB family PTK activity, including cancers and/or other proliferative diseases.

Lapatinib ditosylate salt and its polymorphic forms are disclosed in U.S. Pat. No. 7,157,466. In particular, two polymorphic forms of crystalline lapatinib ditosylate, namely, lapatinib ditosylate monohydrate, anhydrate lapatinib ditosylate (Form I), were disclosed in this publication. Additional anhydrous forms (Form 1, 2, 3, 4, 5 and 6) of crystalline lapatinib ditosylate and solvates were disclosed in WO200907547 and WO 200907541. Form I, II, III, IV, V, VI, VII, VIII, IX, XI, XII, XIII, XIV, XV, XVI, XVII and XVIII of lapatinib ditosylate were disclosed in US2009/0281315.

Since each polymorphic form of a drug substance may display different melting point, hygroscopicity, stability, solubility and/or dissolution rate, crystallinity, crystal habits, bioavailability, toxicity and formulation handling characteristics, which are among the numerous properties that need to be considered in preparing medicament that can be effectively administered. Therefore, the regulatory agencies require a definitive control of one specific polymorphic form of the active component in solid pharmaceutical dosage forms.

Accordingly, there is an ongoing need to search new form of lapatinib ditosylate that may offer advantages for preparing certain desirable pharmaceutical formulations. The novel polymorphic forms of crystalline lapatinib ditosylate in the present invention help fulfill this and other needs.

### SUMMARY OF THE INVENTION

The present inventors have now surprisingly discovered two novel crystalline form of the hydrate ditosylate salt of N-[3-chloro-4-[(3-fluorophenyl) methoxy]phenyl]-6-[5-[[[2-(methylsulfonyl)ethyl]amino]methyl]-2-furanyl]-4-Quinazolinamine (thereafter lapatinib ditosylate or Compound I). Additionally, efficient and reproducible processes are found for the preparation of amorphous form, Form A and Form B of lapatinib ditosylate.

In a first aspect, the present invention is directed to a novel polymorphic form (Form A) of the hydrate ditosylate salt of N-[3-chloro-4-[(3-fluorophenyl) methoxy]phenyl]-6-[5-[[[2-(methylsulfonyl)ethyl]amino]methyl]-2-furanyl]-4-Quinazolinamine.

In a further aspect, the present invention provides a composition comprising (a) Form A of lapatinib ditosylate and (b) a crystalline hydrate, solvate, amorphous, monohydrate, Form 1, 2, 3, 4, 5 and 6 or any other polymorphic forms such as Form I, II, III, IV, V, VI, VII, VIII, IX, XI, XII, XIII, XIV, XV, XVI, XVII and XVIII of lapatinib ditosylate other than Form A, wherein the total weight of lapatinib ditosylate in the composition is the sum of (a) and (b).

In a still aspect, the present invention is directed to a novel polymorphic form (Form B) of the hydrate ditosylate salt of N-[3-chloro-4-[(3-fluorophenyl) methoxy]phenyl]-6-[5-[[[2-(methylsulfonyl)ethyl]amino]methyl]-2-furanyl]-4-Quinazolinamine.

In another aspect, the present invention provides a composition comprising (a) Form B of lapatinib ditosylate and (b) a crystalline hydrate, solvate, amorphous, monohydrate, Form I, II, III, IV, V, VI, VII, VIII, IX, XI, XII, XIII, XIV, XV, XVI, XVII and XVIII, Form 1, 2, 3, 4, 5 and 6 or any other polymorphic forms of lapatinib ditosylate other than Form B, wherein the total weight of lapatinib ditosylate in the composition is the sum of (a) and (b).

In a further aspect, the present invention provides a process for preparing novel polymorph Form A or Form B of lapatinib ditosylate by recrystallizing lapatinib ditosylate in water, a mixture of tetrahydrofuran (THF) and water, followed by isolating and drying the product.

The present invention is further directed to a novel amorphous form of N-[3-chloro-4-[(3-fluorophenyl) methoxy]phenyl]-6-[5-[[[2-(methylsulfonyl)ethyl]amino]methyl]-2-furanyl]-4-Quinazolinamine, bis(4-methylbenzenesulfonate) (lapatinib ditosylate or Compound I).

The amorphous form of the present invention may display distinct characteristics relative to crystalline forms of the N-[3-chloro-4-[(3-fluorophenyl) methoxy]phenyl]-6-[5-[[[2-(methylsulfonyl)ethyl]amino]methyl]-2-furanyl]-4-Quinazolinamine, bis(4-methylbenzenesulfonate), including advantages in the preparation of certain pharmaceutical compositions of Compound I. Amorphous forms of Compound I may also exhibit distinct bioavailability and other pharmacokinetic characteristics compared to known crystalline forms rendering them preferred forms for certain clinical applications.

The present invention further provides a composition and/or drug substance comprising an amorphous form of lapatinib ditosylate in amount from 2% to 100% weight by weight.

The present invention also concerns pharmaceutical compositions comprising the amorphous form of lapatinib ditosylate in amount from 2% to 100% weight by weight with one or more pharmaceutically acceptable excipients.

The present invention further provides a process for preparing the amorphous form of lapatinib ditosylate by using low temperature solvent evaporation technique or high temperature spray solvent removing technique.

The present invention further provides a solid amorphous dispersion of lapatinib ditosylate and a polymer. The said polymer is selected from a group of excipients including starches, polyethylene glycol (PEG) derivatives, polyvinylpyrrolidone (PVP) and co-polymers thereof with PVP or with other polymers, cellulose derivatives such as methylcellulose, hydroxylmethylcelluse, ethylcellulose, hydroxyethylcellulose, hydroxylpropylcellulose, hydroxylpropylmethylcellulose (HPMC), polyacrylates, urea, chitosan and chitosan glutamate, sorbitol or other polyols such as mannitol.

In another aspect, the present invention accordingly provides a pharmaceutical composition comprising Form A, Form B, amorphous form, amorphous solid dispersion of lapatinib ditosylate and one or more pharmaceutically acceptable diluents or carriers and, optionally, one or more other physiologically active agents.

In a sixth aspect, the present invention provides a method for the use of Form A of lapatinib ditosylate for the treatment and/or prophylaxis of patients suffering from disorders characterized by aberrant erB family PTK activity and certain complications thereof.

### BRIEF DESCRIPTION OF THE ACCOMPANYING DRAWINGS

FIG. 1: X-ray powder diffraction (X-RPD) pattern of Form A of lapatinib ditosylate prepared in Example 1.

FIG. 2: X-ray powder diffraction (X-RPD) pattern of Form B of lapatinib ditosylate prepared in Example 2.

FIG. 3: Differential scanning calorimetry (DSC) of Form A of lapatinib ditosylate prepared in Example 1.

FIG. 4: Differential scanning calorimetry (DSC) of Form B of lapatinib ditosylate prepared in Example 2.

FIG. 5: Thermogravimetric analysis (TGA) of Form A of lapatinib ditosylate prepared in Example 1.

FIG. 6: FT-IR spectrum of Form A of lapatinib ditosylate prepared in Example 1.

FIG. 7: X-ray powder diffraction (X-RPD) pattern of amorphous from of lapatinib ditosylate prepared in Example 3.

FIG. 8: Thermogravimetric analysis (TGA) of Form B of lapatinib ditosylate prepared in Example 2.

### DETAILED DESCRIPTION OF THE INVENTION

Unless otherwise indicated, the following definitions are set forth to illustrate and define the meaning and scope of the various terms used to describe the invention herein.

The term “polymorphic form, polymorph, polymorph form, crystalline polymorph or crystalline form of lapatinib ditosylate” in the present invention refers to a crystal modification of lapatinib ditosylate, which can be characterized by analytical methods such as X-ray powder diffraction pattern, Fourier transform infrared spectroscopy (FT-IR), thermogravimetric analysis (TGA), differential scanning calorimetry (DSC), by its melting point or other techniques.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” or “pharmaceutical formulation” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The term “composition” is intended to encompass a particular pure polymorphic (or phase pure) form or a mixture of a particular polymorphic form along with other polymorph forms, solvate, amorphous form, hydrate or co-crystals. The composition may comprise a particular polymorphic form from a trace amount or less than 0.1% to 100% (weight by weight) based on the total amount of lapatinib ditosylate in the composition.

According to one aspect, the present invention provides a novel form of lapatinib ditosylate, e.g., amorphous form of N-[3-chloro-4-[(3-fluorophenyl) methoxy]phenyl]-6-[5-[[[2-(methylsulfonyl)ethyl]amino]methyl]-2-furanyl]-4-Quinazolinamine, bis(4-methylbenzenesulfonate) (lapatinib ditosylate or Compound I). The amorphous form of lapatinib ditosylate herein refers to a solid composition of lapatinib ditosylate or a drug substance of lapatinib ditosylate that comprises more than 2% by weight amorphous lapatinib ditosylate, preferably comprises more than 50% by weight amorphous lapatinib ditosylate, more preferably contains more than 95% by weight (w/w) amorphous lapatinib ditosylate or essentially free of crystalline lapatinib ditosylate.

More specifically, according to one embodiment, the present invention provides the Compound I drug substance that comprises the amorphous form in a detectable amount. By “drug substance” is meant the active pharmaceutical ingredient (API). The amount of the amorphous form in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy. A detectable amount is an amount that can be detected by such physical methods. The limits of detection of such methods are about 2-5% (w/w), and are anticipated to improve with technological advances. The remainder of the drug substance may additionally comprise various crystalline forms of Compound I and polymorphs and pseudopolymorphs (e.g., hydrates or solvates) thereof. In a class of this embodiment, about 2% to about 100% by weight of the amorphous form is present in the drug substance. In a second class of this embodiment, about 10% to about 100% by weight of the amorphous form is present in the drug substance. In a third class of this embodiment, about 25% to about 100% by weight of the amorphous form is present in the drug substance. In a fourth class of this embodiment, about 50% to about 100% by weight of the amorphous form is present in the drug substance. In a fifth class of this embodiment, about 75% to about 100% by weight of the amorphous form is present in the drug substance. In a sixth class of this embodiment, substantially all of the Compound I drug substance is the amorphous form, i.e., the Compound I drug substance is substantially phase pure amorphous form.

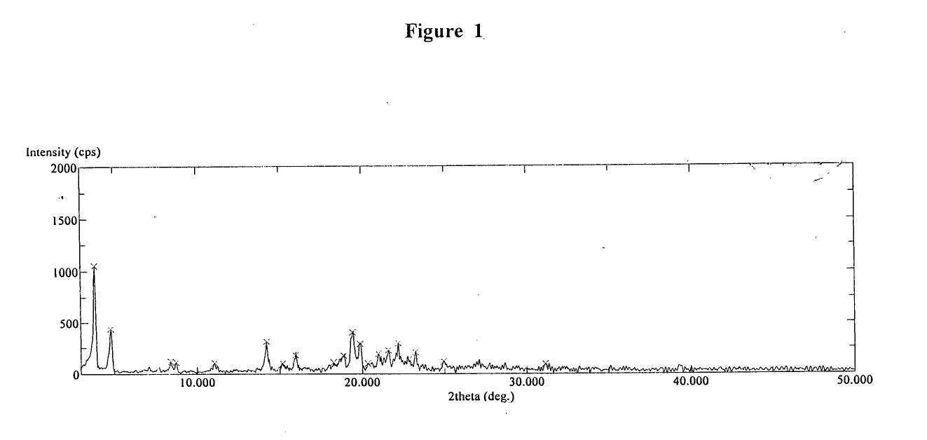

Since the molecule arrangement of amorphous material is in a completely disorder state, the X-ray powder diffraction pattern of amorphous form does not show any discernible or sharp peaks that are characteristics of crystalline material, and only a broad curve is observed, thus demonstrating the amorphous nature of the product. The amorphous form of lapatinib ditosylate characterized by X-ray powder diffraction pattern is essentially identical to the accompanied drawing of FIG. 1 in the present invention.

The amorphous form of Compound I according to the present invention further includes anhydrous amorphous form of Compound I, hydrate amorphous form of Compound I or solvate amorphous form of lapatinib ditosylate.

According to another aspect, the present invention provides two processes for preparing amorphous form of N-[3-chloro-4-[(3-fluorophenyl) methoxy]phenyl]-6-[5-[[[2-(methylsulfonyl)ethyl]amino]methyl]-2-furanyl]-4-Quinazolinamine, bis(4-methylbenzenesulfonate) (lapatinib ditosylate or Compound I).

Accordingly, the first process for preparing amorphous form of lapatinib ditosylate is a low temperature solvent evaporation process, including the following steps:
a) dissolving lapatinib ditosylate in a mixture of water and acetone, tetrahydrofuran (THF), acetonitrile or a straight or branched chain C₁-C₄ alcohol solvent or their mixture of any two solvents thereof,
b) freezing the solution to obtain frozen solid substance under a low temperature,
c) removing solvents from the frozen solid under vacuum to obtain amorphous lapatinib ditosylate.

More specifically, in a first step of the process, lapatinib ditosylate is preferably dissolved in an aqueous solvent, more preferably dissolved in an aqueous alcohol co-solvent, and most preferably dissolved in a mixture of water and ethanol or methanol to form a solution.

In particular, lapatinib ditosylate is moderately soluble in a mixture of water and methanol or ethanol, allowing the complete dissolution of lapatinib ditosylate at room temperature at a concentration of about 5 mg or above 5 mg per milliliter (mL). The use of a relatively concentrated solution, e.g. about 5 mg/mL or above 5 mg/mL therefore is preferred.

In a second and preferred step of the process, a solution of lapatinib ditosylate in a solvent is solidified to allow the solution become a solid residue containing lapatinib ditosylate in an amorphous state.

In the first stage of solidifization, the temperature of the solution is decreased until the solution is completely frozen or becomes a solid, typically to temperatures as low as minus 50° C., and below, to produce a frozen solid mixture. Such cooling allows the solute and solvent to separate into separate solid phases. Usually, phase separation will yield a solute in an amorphous state, but may also yield crystalline, microcrystalline or their mixtures. Preferably in this invention, cooling is performed rapidly so that the formation of solute crystals is inhibited, and only amorphous material is formed. More preferably, the solution is cooled using liquid nitrogen with swirling of the vessel containing the solution to coat the wall of the vessel and accelerate freezing. Once the solution has been completely frozen, it is then possible to remove the separated solvent from the frozen mixture by warming up the contents slowly so that the solvent evaporates from the frozen mixture through sublimation under the reduced pressure, preferably under vacuum.

Therefore, the solvent evaporation step is preferably conducted under vacuum so that the frozen solvent will vaporize without melting. This vapor migrates through the frozen mixture and escapes into the evacuated space outside of the frozen mixture. The vapor is re-condensed on a refrigerated surface, and turns into a liquid in condenser. The condenser is maintained at a temperature below that of the frozen solid mixture to drive the solvent evaporation process.

When the solvent is a mixture of water and alcohol solvent, typical frozen solid formation conditions for producing amorphous form of lapatinib ditosylate include that the low temperature of the frozen solid mixture is from about −85° C. to about −0° C. before vacuum is applied. The vacuum is typically about 0.05 mm Hg or less, more preferably about 0.01 mm Hg or less and the temperature of the frozen solid mixture is from about −85° C. to about 10° C. during the solvent removing stage. The solvent evaporation time using these conditions and standard equipment is dependent on the amount and the nature of solute and solvent used. The solvent evaporation time is from about 24 hours to about 96 hours for about a 50 g sample of lapatinib ditosylate dissolved in water and ethanol.

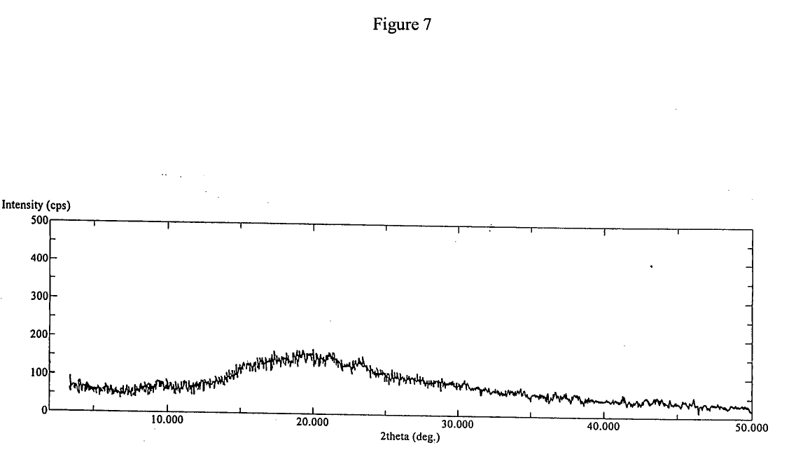

The obtained product prepared according to the process of the present invention may be characterized by X-ray powder diffraction pattern, as shown in the accompanied drawing of FIG. 7. The X-ray powder diffraction pattern of amorphous form of lapatinib ditosylate obtained in the present invention does not show any discernible or sharp peaks that are characteristic of crystalline materials; only a flat or broad curve is observed, thus demonstrating the amorphous nature of the product.

The straight or branched chain C₁-C₄ alcohol solvent in the present invention is selected from the group of methanol, ethanol, n-propanol, isopropanol or branched-chain butanols, preferably the alcohol solvent is methanol, ethanol or mixtures thereof. The processes can be carried out with two or more alcohol solvents.

The amorphous form of lapatinib ditosylate obtained from above process can be anhydrous amorphous lapatinib ditosylate or hydrate amorphous lapatinib ditosylate or amorphous solvate lapatinib ditosylate.

In a further aspect, the current invention provides a high temperature spray drying solvent removing process for preparing the amorphous lapatinib ditosylate, including following steps: a) dissolving the lapatinib ditosylate in a mixture of water and a straight or branched chain C₁-C₄ alcohol solvent or their mixture of any two solvents thereof; b) stirring the solution until it becomes clear; c) removing the solvent by a high temperature spray drying solvent method; d) further solvent removing the product under vacuum at elevated temperature until loss of solvent removing is less than 0.5% or constant.

The high temperature spray drying solvent removing process can be carried out using any commercially available spray dryers, which are used, operates on the principle of nozzle spraying in a parallel flow. For instance, the sprayed product and solvent removing gas flow in the same direction. The solvent removing gas can be air or inert gasses such as nitrogen, argon and carbon dioxide. Nitrogen gas is preferred in this invention. For lapatinib ditosylate and polymer solution, the spray solvent removing in-let temperature is about 140-180° C., and the out-let temperature is about 90-60° C. at a feed rate of 5-25 ml/min.

Specifically, the crude lapatinib ditosylate or pure lapatinib ditosylate are usually dissolved in a mixture of water and alcohol solvent, such as aqueous methanol or aqueous ethanol. The concentration of lapatinib ditosylate is from 1% to 20% (w/v), preferably from 2% to 15%. If necessary, the solution can be heated to completely dissolve the starting materials, and then the solvent is removed by spray process to obtain the solid product. The solution is cooled to 30° C., and then proceeds with spray solvent removing procedure.

The product obtained from spray solvent removing method is further dried to remove the solvent. The product can be dried in a tray drier or dried under vacuum or in a Fluid Bed Dryer. The drying temperature is preferably from 20 to 70° C., the drying time is preferably from 8-24 hours. The most preferred drying temperature 35-40° C. and drying time is 12 to 48 hours. After drying, the obtained solid product is the amorphous lapatinib ditosylate.

Amorphous lapatinib ditosylate can be obtained using this simple and reproducible spray solvent removing process.

According to a process of the invention, the starting material of lapatinib ditosylate can be obtained by any methods described in U.S. Pat. No. 7,157,466. The starting material lapatinib ditosylate can be crude or pure lapatinib ditosylate, including any solvates or hydrates, preferably purity is more than 95%, more preferably purity is more than 98%, most preferably purity is more than 99%. Starting material lapatinib ditosylate can be any polymorph forms, including amorphous or crystalline form or their mixture thereof. With the processes where lapatinib ditosylate goes into solution, the form of the starting material is of minimal relevance since any solid-state structure is lost in solution.

Amorphous lapatinib ditosylate prepared according to the process of the present invention may be characterized by X-ray powder diffraction pattern, as shown in the accompanied drawing of FIG. 7. The X-ray powder diffraction pattern of amorphous lapatinib ditosylate does not show any discernible peaks that are characteristic of crystalline materials. The lack of discernible or sharp peaks indicates the characteristic feature of amorphous lapatinib ditosylate, and also demonstrating the amorphous nature of the obtained product.

The amorphous form of lapatinib ditosylate prepared according to the procedures of the present invention can be used to make pharmaceutical compositions. Therefore, the present invention further provides a pharmaceutical composition for administering effective amount of amorphous form of lapatinib ditosylate as active ingredient in unit dosage forms. The unit dosage forms can be administered in a wide variety of oral and parenteral dosage forms, such as by injection, that is, intravenously or intramuscularly. Also, the amorphous form of lapatinib ditosylate of the present invention can be administered by inhalation, e.g. intranasally or transdermally. It will be obvious to those skilled in the art that the following dosage forms may comprise either amorphous form of lapatinib ditosylate, as the active component.

More specifically, the present invention also provides pharmaceutical compositions comprising the amorphous Compound I, in association with one or more pharmaceutically acceptable polymers or excipients. In one embodiment the pharmaceutical composition comprises a prophylactically or therapeutically effective amount of lapatinib ditosylate as the active pharmaceutical ingredient (API) in admixture with pharmaceutically acceptable excipients wherein the API comprises a detectable amount of the amorphous form of the present invention. In a second embodiment the pharmaceutical composition comprises a prophylactically or therapeutically effective amount of lapatinib ditosylate as the API in admixture with pharmaceutically acceptable excipients wherein the API comprises about 2% to about 100% by weight of amorphous Compound I of the present invention. In a class of this second embodiment, the API in such compositions comprises about 10% to about 100% by weight of amorphous Compound I. In a second class of this embodiment, the API in such compositions comprises about 25% to about 100% by weight of amorphous Compound I. In a third class of this embodiment, the API in such compositions comprises about 50% to about 100% by weight of amorphous Compound I. In a fourth class of this embodiment, the API in such compositions comprises about 75% to about 100% by weight of amorphous Compound I. In a fifth class of this embodiment, substantially the entire API is amorphous Compound I, i.e., the API is substantially phase pure amorphous Compound I. When not comprising substantially phase pure amorphous Compound I, such compositions may additionally comprise various crystalline forms of Compound I and polymorphs and pseudopolymorphs thereof.

In a still aspect, the present invention further provides a solid amorphous dispersion of lapatinib ditosylate and a polymer or a mixture of one or more polymers. For solid amorphous dispersion of lapatinib ditosylate and a polymer, the composition comprises more 2% of the amorphous form of lapatinib ditosylate, preferably more than 50% the amorphous form of lapatinib ditosylate, most preferably more than 95% or substantially free of any crystalline forms other than its amorphous form.

The solid amorphous dispersion of the present invention has the following characteristics. The amorphous lapatinib ditosylate is evenly dispersed in the polymer. This highly dispersed material does not contain any crystalline substance, and therefore, it will not induce the crystallization of amorphous material to become a crystalline material. That is, since the amorphous lapatinib ditosylate is highly dispersed in the amorphous polymer, it will not convert back into crystalline lapatinib ditosylate. The solid amorphous dispersion of lapatinib ditosylate is also stable, and has a good material flow property and high bulk & tap density, and thus it is particularly suitable for preparation of pharmaceutical composition.

The polymer used to make the solid amorphous dispersion of the present invention may be an amorphous material or it can be converted into amorphous material. The suitable polymers should be soluble in water, methanol, ethanol, acetonitrile, acetone and dichloromethane or mixtures thereof. The suitable polymers should be pharmaceutically acceptable as well.

The suitable polymers of the present invention are the cellulose derivatives, which is selected from hydroxy-propylcellulose (HPC), hydroxypropylmethylcellulose (HPMC) and hydroxypropyl-methylcellulose acetate succinate (HPMC-AS). The solid dispersion in such case comprises preferably between 50 and 90% by weight of the cellulose derivative and 10 to 50% by weight of amorphous lapatinib ditosylate.

The suitable polymers of the present invention include polyvinylpyrrolidone derivatives, which is selected from polyvinylpyrrolidone (PVP) and co-polymers thereof with PVP. The preferred polyvinylpyrrolidone derivative is PVP, cross-linked PVP, PVP-VA64. The solid dispersion preferably comprises between 50 and 90% by weight of PVP derivative and 10 to 50% by weight of amorphous lapatinib ditosylate.

The suitable polymers of the present invention include polyethyleneglycols, which are selected from Polyethyleneglycol 8000 and Polyethyleneglycol 6000. The solid dispersion preferably comprises between 50 and 90% by weight of a polyethyleneglycol and 10 to 50% by weight of amorphous lapatinib ditosylate.

The suitable polymer of the present invention includes polyethylene-/polypropylene-/polyethylene-oxide block copolymer. The preferred such copolymer is Pluronic F68. The solid dispersion preferably comprises between 50 and 90% by weight of a polyethylene-/polypropylene-/polyethylene-oxide block copolymer and 10 to 50% by weight of amorphous lapatinib ditosylate.

Eudragit®L-1 00-55 and Eudragit®E-100 are also suitable polymethacrylates for the present invention. The solid dispersion preferably comprises between 50 and 90% by weight of a polymethacrylates and 10 to 50% by weight of amorphous lapatinib ditosylate.

The solid dispersions may contain amorphous lapatinib ditosylate in an amount by weight of the composition of about 0.01% to about 80%; for example, in an amount by weight of about 0.01% to about 80%, 0.1% to about 70%, such as 1% to 60%, for example 2%, 5%, 10%, 20%, 30%, 40%, 50%, or 60%. The polymeric excipient may be present in an amount from about 0.1% to 99.99% by weight of the composition.

Additional suitable polymers to make the solid amorphous dispersion of the present invention include α-cyclodextrin, β-cyclodextrin and its derivatives, hydroxylpropyl-β-cyclodextrin and its derivatives, preferably hydroxylpropyl-β-cyclodextrin and its derivatives.

The suitable polymers to make the solid amorphous dispersion of the present invention should be commercially available or can be made by known procedures.

According to another aspect, the present invention provides a process for preparing a solid amorphous dispersion of lapatinib ditosylate and a polymer using vacuum distillation or spray solvent removing technique, and the details can be found in examples of the present inventions. Vacuum distillation technique for preparing solid amorphous dispersion includes the following steps: dissolving the starting materials of lapatinib ditosylate in a mixture of water and solvent, e.g. methanol, ethanol, acetone, acetonitrile or mixtures thereof at heating, preferably heated to boiling point of the solvent; evaporating the solvent under the reduced pressure or vacuum to dryness; grinding the solid residues; and further drying the product under vacuum at 35-45° C. Spray solvent removing technique for preparing solid amorphous dispersion comprises the following steps: dissolving the starting material of lapatinib ditosylate in a mixture of water and solvent, e.g. methanol, ethanol, acetone, acetonitrile or mixtures of solvents thereof at heating; cooling the solution to 30° C.; removing the solvent by spray drying solvent removing method to afford solid residues; further drying the product under vacuum at 25-50° C. Alternatively, the lapatinib ditosylate and polymer solution can be prepared by separately dissolving lapatinib ditosylate or polymer in a solvent to give individual solution, and then lapatinib ditosylate solution can be added into the polymer solution or vice versa to afford a solution of lapatinib ditosylate and polymer.

The solid amorphous dispersion of lapatinib ditosylate and a polymer and pharmaceutically acceptable excipients can be used to prepare pharmaceutical compositions. Therefore, according to a further aspect, the present invention provides a pharmaceutical composition for administering effective amount of amorphous lapatinib ditosylate in a form of solid amorphous dispersion in unit dosage forms. The unit dosage forms can be administered in a wide variety of oral and parenteral dosage forms as described above. Additionally, it will be obvious to those skilled in the art that the following dosage forms may comprise either amorphous lapatinib ditosylate or a corresponding pharmaceutically acceptable salt of a compound of the present invention as the active component.

Amorphous lapatinib ditosylate in unit dosage form may comprise 10-800 mg, preferably 100-600 mg, most preferably 200-500 mg, as active ingredient.

The particle sizes of amorphous form of lapatinib ditosylate of the present invention is about 1-400 μm, preferably about 5-250 μm, more preferably about 10-200 μm, most preferably about 50-150 μm. Small particle sizes are better in improving the blending uniformity or content uniformity of the unit dosage forms such as tablets, particularly in direct compression process.

The unit dosage forms of pharmaceutical composition comprising amorphous lapatinib ditosylate or its solid amorphous dispersions can be fast, immediate release or sustained release products, and they can be prepared according to the conventional procedures used in pharmaceutical industry. The details for preparation of tablets are described in Example 5 of the present invention.

According to one aspect of the present invention, there is provided a novel polymorphic form of the hydrate ditosylate salt of N-[3-chloro-4-[(3-fluorophenyl) methoxy]phenyl]-6-[5-[[[2-(methylsulfonyl)ethyl]amino]methyl]-2-furanyl]-4-Quinazolinamine (thereafter lipatinib ditosylate), designated as Form A herein, having an X-ray powder diffraction pattern (X-RPD), or substantially the same X-ray powder diffraction pattern, as shown in FIG. 1. More particularly, polymorphic Form A of lapatinib ditosylate according to the present invention can be characterized as having an X-ray diffraction pattern with characteristic peaks (expressed in 2θ±0.2° 2θ) at one or more of the following positions: 3.86, 4.80, 14.2, 15.96, 19.42 or 19.88. Form A of lapatinib ditosylate according to the present invention can be further characterized as having an X-ray diffraction pattern with characteristic peaks (expressed in 2θ±0.2° 2θ) at one or more of the following positions: 3.86, 4.80, 8.40, 8.74, 14.2, 15.96, 18.26, 18.88, 19.42, 19.88, 20.34, 20.98, 21.58, 22.18, 22.80, 23.22 or 24.94.

Characterizing data for crystalline Form A of lapatinib ditosylate according to the present invention as obtained by X-ray powder diffraction is substantially the same as shown in FIG. 1 and Table 1.

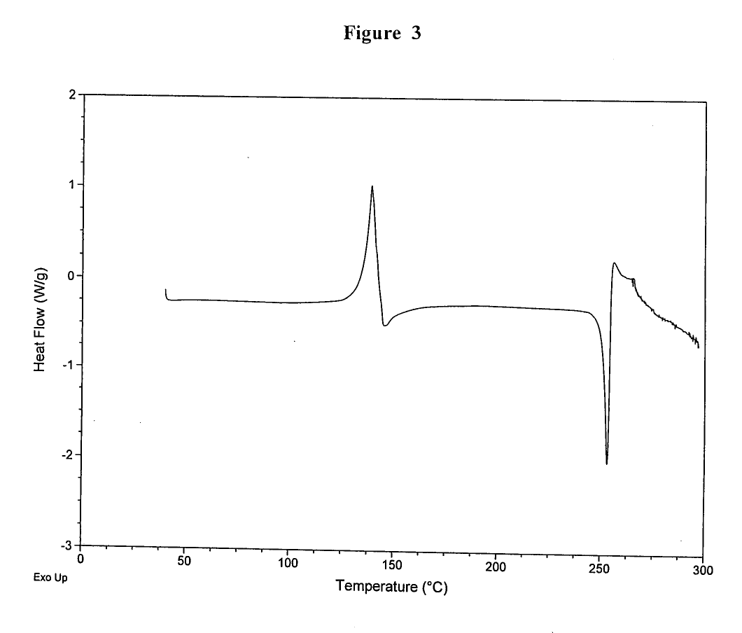

Further characterizing data for polymorph Form A of lapatinib ditosylate according to the present invention as obtained by differential scanning calorimetry (DSC) is substantially the same as shown in FIG. 3, and it provides an exothermic peak at around 137-142° C. (typically 139° C.) and an endothermic peak at around 251-255° C. (typically about 253° C.).

TABLE 1
Characteristic X-ray Powder Diffraction Pattern Peaks
(expressed in 2θ ± 0.2° 2θ) and Relative
Intensities of Diffraction Lines for Form A of Lapatinib ditosylate

| Degree 2θ ± 0.2° 2θ | I/Iₒ |
|---|---|
| 3.86 | 100 |
| 4.80 | 42 |
| 8.40 | 12 |
| 8.74 | 11 |
| 14.2 | 30 |
| 15.96 | 17 |
| 18.26 | 11 |
| 18.88 | 17 |
| 19.24 | 39 |
| 19.88 | 28 |
| 20.34 | 10 |
| 20.98 | 19 |
| 21.58 | 22 |
| 22.18 | 29 |
| 22.80 | 13 |
| 23.22 | 20 |
| 24.94 | 12 |

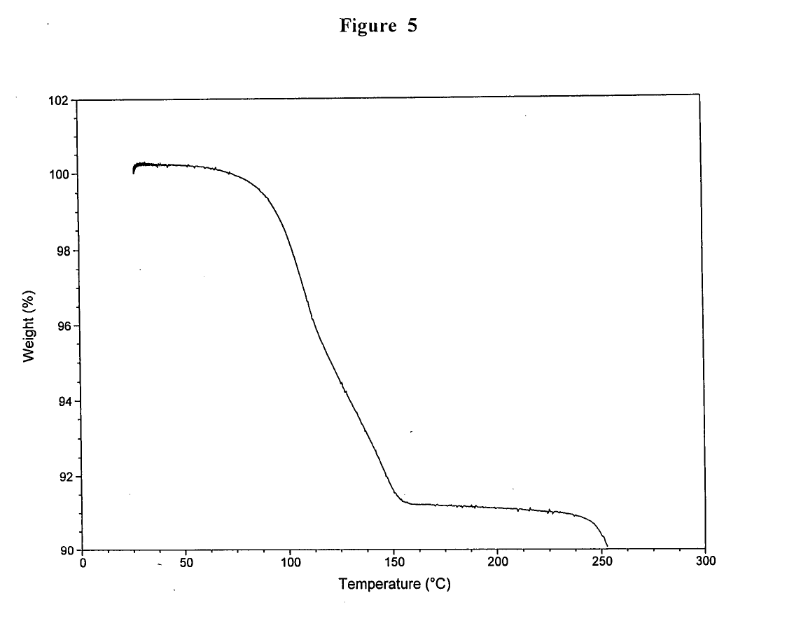

Still further characterizing data for polymorphic Form A of lapatinib ditosylate according to the present invention obtained by thermogravimetric analysis (TGA) is substantially the same as shown in FIG. 5, and it provides a loss of water at about 9.0% w/w from about 65° C. to about 150° C. The lapatinib ditosylate monohydrate would theoretically provide a loss of water at about 1.92% w/w. The lapatinib ditosylate monohydrate disclosed in WO02/02552 contains 1.99% w/w water (refer to Example 10 in WO02/02552). X-RPD, DSC and TGA data of Form A of lapatinib ditosylate according to the present invention is clearly different from that of lapatinib ditosyale monohydrate disclosed in WO02/02552. Therefore, Form A of the present invention is a new and novel polymorphic form of lapatinib ditosylate, and furthermore Form A is a new and novel form of lapatinib ditosylate hydrate.

In a preferred aspect, Form A according to the present invention is the hydrate form of lapatinib ditosylate, or the hydrate ditosylate salt of lapatinib, wherein two to seven water molecules, preferably two to five water molecules, more preferably three to four water molecules, most preferably four and half water molecules co-exist or preferably co-crystallize with one lapatinib ditosylate salt molecule. Alternatively, the molar ratio of lapatinib:tosylate:water in Form A is 1:2:2-7, preferably 1:2:2-6, more preferably 1:2:3-5, and most preferably 1:2:3-4, as shown in the following chemical structure and formula:

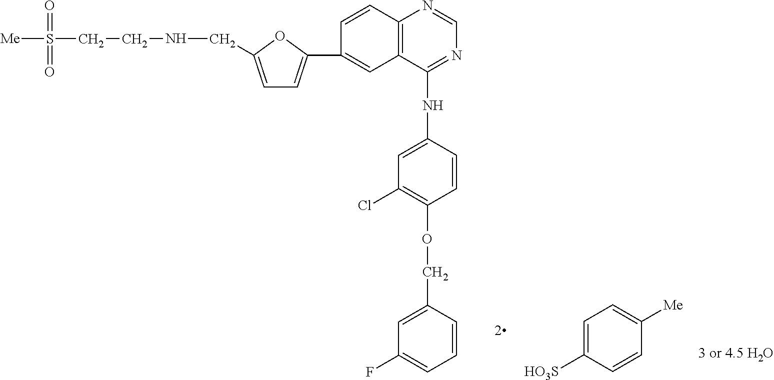

The crystalline form of lapatinib ditosylate hydrate (Form A) according to the present invention is thermally stable form. For instance, Form A does not undergo a phase transformation even heating up to 40° C. Additionally, Form A has good material flow characteristic and adequate chemical stability. These favorable characters render Form A a superior polymorphic form for pharmaceutical formulation and bulk handling of lapatinib ditosylate.

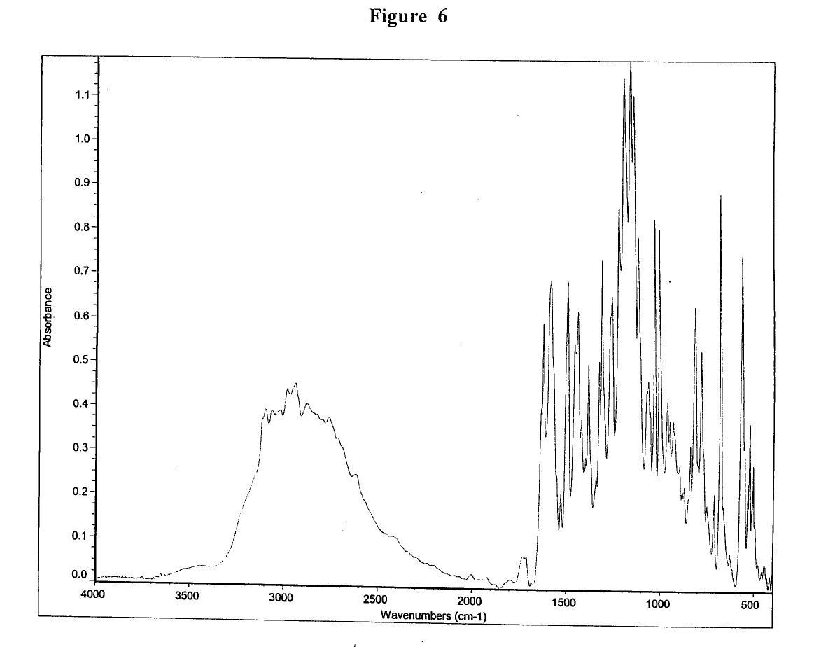

Further characterizing data for polymorphic Form A of lapatinib ditosylate according to the present invention obtained by the Fourier transform infrared (FT-IR) spectrum is substantially the same as shown in FIG. 6, and it contains peaks at one or more of the following positions of about 2939, 1622, 1583, 1528, 1495, 1441, 1384, 1327, 1313, 1260, 1224, 1198, 1164, 1147, 1121, 1067, 1033, 1009, 965, 952, 933, 840, 817, 783, 711, 681, 565, 524, 532 or 505 cm⁻¹.

In one favored aspect, the polymorph Form A of lapatinib ditosylate provides X-ray powder diffraction (X-RPD) pattern substantially in accordance with FIG. 1 and Table 1.

In one favored aspect, the polymorph Form A of lapatinib ditosylate provides differential scanning calorimetry (DSC) substantially in accordance with FIG. 3.

In one still favored aspect, the Form A of lapatinib ditosylate provides thermogravimetric analysis (TGA) substantially in accordance with FIG. 5.

In one favored aspect, the Form A of lapatinib ditosylate provides the Fourier transform infrared (FT-IR) substantially in accordance with FIG. 6.

The present invention encompasses Form A of lapatinib ditosylate isolated in pure form or in a mixture as a solid composition when admixed with other materials, for example the other known polymorphic forms (i.e. amorphous form, solvates, monohydrate, Form I, II, III, IV, V, VI, VII, VIII, a, XI, XII, XIII, XIV, XV, XVI, XVII and XVIII, Form 1, 2, 3, 4, 5 and 6, or other forms of lapatinib ditosylate or any other materials.

Thus in one aspect there is provided Form A of crystalline lapatinib ditosylate in isolated solid form.

In a further aspect there is provided Form A of lapatinib ditosylate in phase pure form. The phase pure form means that Form A is over 95% (w/w), preferably over 98% (w/w), more preferably over 99% (w/w %) and most preferably over 99.5% (w/w) or over 99.9% (w/w).

More specifically, the present invention provides that Form A of lapatinib ditosylate is in the form of a composition or a mixture of Form A along with one or more other crystalline, solvate, amorphous, or other polymorphic forms or their combinations thereof of lapatinib ditosylate. Such a composition may be a drug substance or an active ingredient in pharmaceutical compositions or formulations. For example, such composition may comprise polymorphic Form A along with one or more other polymorphic forms of lapatinib ditosylate, such as amorphous form, hydrate, solvates, monohydrate, Form I, II, III, IV, V, VI, VII, VIII, IX, XI, XII, XIII, XIV, XV, XVI, XVII and XVIII, Form 1, 2, 3, 4, 5 and 6, or other polymorphic forms or their combinations thereof. More specifically, the composition may comprise from trace amounts up to 100% Form A, or any amount in between—for example, the composition may comprise less than 0.1%, 0.5%, 1%, 2%, 5%, 10%, 20%, 30%, 40% or 50% by weight of Form A based on the total amount of lapatinib ditosylate in the composition. Alternatively, the composition may comprise at least 50%, 60%, 70%, 80%, 90%, 95%, 97%, 98%, 99%, 99.5% or 99.9% by weight of Form A based on the total amount of lapatinib ditosylate in the composition.

In yet a further aspect there is provided Form A of lapatinib ditosylate in crystalline form.

In a preferred aspect, the particle size of polymorphic Form A of lapatinib ditosylate in the present invention has the median value of the volume mean diameter of the particles within the range of 0.01 μm-450 μm, preferably 5-250 μm, and most preferably 50-150 μm. Such particles are better in chemical and physical stability, good material flow characteristics, improving the uniformity of dosage forms and thus suitable for bulk preparation and formulation advantages.

According to another aspect, the present invention provides a process for preparing polymorph Form A of lapatinib ditosylate. Polymorph Form A may be prepared by crystallization from a crystallization solvent containing lapatinib ditosylate. As used herein, the term “crystallization solvent” means a solvent or combination of solvents from which lapatinib ditosylate is preferentially crystallized as polymorph Form A. Representative crystallization solvents for preparation of Form A include water, tetrahydrofuran (THF) and combinations thereof. In a preferred aspect, the crystallization solvent comprises tetrahydrofuran (THF), to which water is gradually added.

In a preferred aspect, Form A of lapatinib ditosylate may be prepared by slurring starting material, crude or pure lapatinib ditosylate, anhydrate or solvate, which can be obtained according to the procedures described in U.S. Pat. No. 7,157,466 (or WO02/02552) with an organic solvent or a mixture of two or more organic solvents under heat. The lapatinib ditosylate is soluble in a mixture of water and THF, but not soluble in non-polar organic solvents. The preferred organic solvent is tetrahydrofuran. The concentration of lapatinib ditosylate within the solution may range from about 0.1% by weight to the saturation point. This concentration will, of course, vary depending upon the temperature at which the co-solvent solution is held, with warmer temperatures generally allowing for the preparation of more concentrated solutions of lapatinib ditosylate. Preferably, the concentration (w/w %) of lapatinib ditosylate starting material in solution is about 0.5-15%, preferably about 1-10%, more preferably about 1.5-5%. The volume ratio of tetrahydrofuran to water is about 50-95:50-5, preferably about 70-95:30-5, more preferably about 80-95:20-5, most preferably about 85:15. Water is then added into the above suspension, and the mixture is heated, suitably to a temperature in the range of from about 45° C. to 85° C., such as about 50° C. to 75° C., for example about 65-70° C. until all solid materials are dissolved. The clear and hot solution is allowed to cool down to ambient temperature, and the cooled solution is kept at ambient temperature for crystallization. The crystal Form A of lapatinib ditosylate is formed over a period of 1 hour to 4 days, and the crystal Form A is recovered from the solvent by filtration. The obtained crystal From A can be dried under a vacuum oven at about 20° C.-60° C., preferably at about 25° C.-50° C., more preferably at about 25° C.-35° C., and most preferably at 30-40° C. for about 10-40 hours to remove the solvent residues.

Once obtained, crystals of polymorph Form A may be used as the nucleating agent or “seed” crystals for subsequent crystallizations of polymorph Form A from the crystallization solvent. In one embodiment, the crystallization solvent is formed by dissolving lapatinib ditosylate in hot tetrahydrofuran and water or other suitable crystallization solvents. The crystallization solvent is then seeded with crystals of polymorph Form A, cooled and filtered, resulting in polymorph Form A. In another embodiment, a crystallization solvent is formed by slurring lapatinib ditosylate in tetrahydrofuran and water or other appropriate solvents. The crystallization solvent is then seeded with crystals of polymorph Form A and filtered, resulting in polymorph Form A. Such seeding with crystals of polymorph Form A may take place at any time during the slurring process. Alternatively, seeding with crystals of polymorph Form A may take place prior to, or simultaneously with, addition of lapatinib ditosylate to the crystallization solvent.

Form A of crystalline lapatinib ditosylate as obtained above is characterized by X-ray powder diffraction pattern, substantially the same as shown in FIG. 1 and Table 1.

Form A of lapatinib ditosylate as obtained above is characterized by differential scanning calorimetry (DSC), substantially the same as shown in FIG. 3.

The crystals of lapatinib ditosylate obtained from recrystallization in solvents as described in above processes may have different crystal habits (e.g., shape), water contents, surface area, bulk or tap density, or particle size, but they clearly still belong to a new and novel polymorphic form (Form A) of lapatinib ditosylate, as it is characterized and confirmed by X-ray powder diffraction pattern and DSC thermogram, TGA and FT-IR. The X-ray powder diffraction pattern of Form A is clearly different from that of other known forms such as lapatinib ditosylate monohydrate, Form 1, 2, 3, 4, 5 and 6 or other known polymorph forms or solvates.

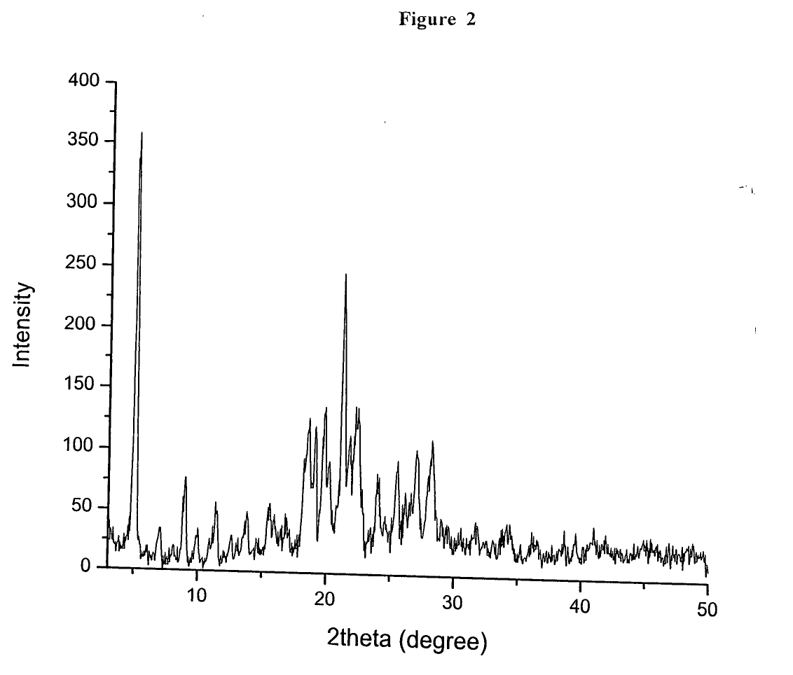

According to one aspect of the present invention, there is provided a novel polymorphic form of the ditosylate salt of N-[3-chloro-4-[(3-fluorophenyl) methoxy]phenyl]-6-[5-[[[2-(methylsulfonyl)ethyl]amino]methyl]-2-furanyl]-4-Quinazolinamine (thereafter lipatinib ditosylate), designated as Form B herein, having an X-ray powder diffraction pattern (X-RPD), or substantially the same X-ray powder diffraction pattern, as shown in FIG. 2. More particularly, polymorphic Form B of lapatinib ditosylate according to the present invention can be characterized as having an X-ray diffraction pattern with characteristic peaks (expressed in 2θ±0.2° 2θ) at one or more of the following positions: 5.10, 9.00, 15.57, 19.10, 19.85, 21.20, 22.20, 27.00 or 28.25. Form B of lapatinib ditosylate according to the present invention can be further characterized as having an X-ray diffraction pattern with characteristic peaks (expressed in 2θ±0.2° 2θ) at one or more of the following positions: 5.10, 7.05, 9.00, 10.00, 11.50, 13.80, 15.57, 19.10, 19.85, 21.20, 22.20, 24.60, 25.55, 27.00 or 28.25.

Characterizing data for crystalline Form B of lapatinib ditosylate according to the present invention as obtained by X-ray powder diffraction is substantially the same as shown in FIG. 2 and Table 2.

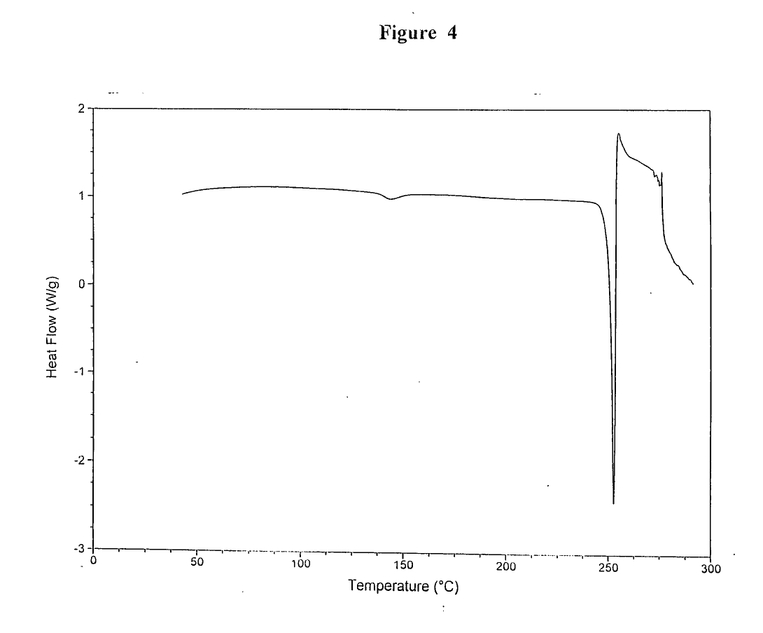

Further characterizing data for polymorph Form B of lapatinib ditosylate according to the present invention as obtained by differential scanning calorimetry (DSC) is substantially the same as shown in FIG. 4 and it provides an endothermic peak at around 142-145° C. (typically 144° C.) and an endothermic peak at around 251-255° C. (typically about 253° C.).

TABLE 2
Characteristic X-ray Powder Diffraction Pattern Peaks
(expressed in 2θ ± 0.2° 2θ) and Relative
Intensities of Diffraction Lines for Form A of Lapatinib ditosylate

| Degree 2θ ± 0.2° 2θ | I/Iₒ |
|---|---|
| 5.10 | 100 |
| 7.05 | 9 |
| 9.00 | 22 |
| 10.00 | 10 |
| 11.50 | 14 |
| 13.80 | 14 |
| 15.57 | 16 |
| 19.10 | 34 |
| 19.85 | 38 |
| 21.20 | 69 |
| 22.20 | 26 |
| 24.60 | 7 |
| 25.55 | 27 |
| 27.00 | 29 |
| 28.25 | 30 |

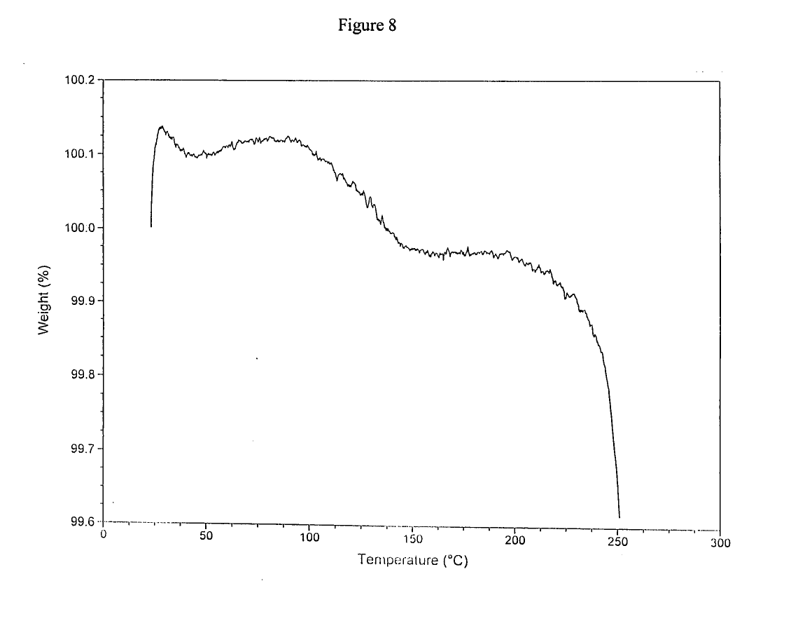

Still further characterizing data for polymorphic Form B of lapatinib ditosylate according to the present invention obtained by thermogravimetric analysis (TGA) is substantially the same as shown in FIG. 8, and it provides a loss of water at about less than 0.2% w/w from about 65° C. to about 150° C. The lapatinib ditosylate monohydrate would theoretically provide a loss of water at about 1.92% w/w. The lapatinib ditosylate monohydrate disclosed in WO02/02552 contains 1.99% w/w water (refer to Example 10 in WO02/02552). X-RPD, DSC and TGA data of Form B of lapatinib ditosylate according to the present invention is clearly different from that of lapatinib ditosyale monohydrate and anhydrate form (Form I) disclosed in WO02/02552, Form I, II, III, IV, V, VI, VII, VIII, IX, XI, XII, XIII, XIV, XV, XVI, XVII and XVIII disclosed in US2009/0281315, and Form 1, 2, 3, 4, 5 and 6 disclosed in WO2009/079541. Therefore, Form B of the present invention is a new and novel polymorphic form of lapatinib ditosylate, and furthermore Form B is a new and novel form of lapatinib ditosylate anhydrate.

The crystalline form of lapatinib ditosylate (Form B) according to the present invention is thermally stable form. For instance, Form B does not undergo a phase transformation even heating up to 45° C. Additionally, Form B has good material flow characteristic and adequate chemical stability. These favorable characters render Form B a superior polymorphic form for pharmaceutical formulation and bulk handling of lapatinib ditosylate.

In one favored aspect, the polymorph Form B of lapatinib ditosylate provides X-ray powder diffraction (X-RPD) pattern substantially in accordance with FIG. 2 and Table 2.

In one favored aspect, the polymorph Form B of lapatinib ditosylate provides differential scanning calorimetry (DSC) substantially in accordance with FIG. 4.

In one still favored aspect, the Form B of lapatinib ditosylate provides thermogravimetric analysis (TGA) substantially in accordance with FIG. 8.

The present invention encompasses Form B of lapatinib ditosylate isolated in pure form or in a mixture as a solid composition when admixed with other materials, for example the other known polymorphic forms (i.e. amorphous form, solvates, monohydrate, Form I, Form 1, 2, 3, 4, 5 and 6, or other forms of lapatinib ditosylate or any other materials.

Thus in one aspect there is provided Form B of crystalline lapatinib ditosylate in isolated solid form.

In a further aspect there is provided Form B of lapatinib ditosylate in phase pure form. The phase pure form means that Form B is over 95% (w/w), preferably over 98% (w/w), more preferably over 99% (w/w %) and most preferably over 99.5% (w/w) or over 99.9% (w/w).

More specifically, the present invention provides that Form B of lapatinib ditosylate is in the form of a composition or a mixture of Form B along with one or more other crystalline, solvate, amorphous, or other polymorphic forms or their combinations thereof of lapatinib ditosylate. Such a composition may be a drug substance or an active ingredient in pharmaceutical compositions or formulations. For example, such composition may comprise polymorphic Form B along with one or more other polymorphic forms of lapatinib ditosylate, such as amorphous form, hydrate, solvates, monohydrate, Form I, II, III, IV, V, VI, VII, VIII, IX, XI, XII, XIII, XIV, XV, XVI, XVII and XVIII, Form 1, 2, 3, 4, 5 and 6, or other polymorphic forms or their combinations thereof. More specifically, the composition may comprise from trace amounts up to 100% Form B, or any amount in between—for example, the composition may comprise less than 0.1%, 0.5%, 1%, 2%, 5%, 10%, 20%, 30%, 40% or 50% by weight of Form B based on the total amount of lapatinib ditosylate in the composition. Alternatively, the composition may comprise at least 50%, 60%, 70%, 80%, 90%, 95%, 97%, 98%, 99%, 99.5% or 99.9% by weight of Form B based on the total amount of lapatinib ditosylate in the composition.

In yet a further aspect there is provided Form B of lapatinib ditosylate in crystalline form.

In a preferred aspect, the particle size of polymorphic Form B of lapatinib ditosylate in the present invention has the median value of the volume mean diameter of the particles within the range of 0.01 μm-450 μm, preferably 5-250 μm, and most preferably 50-150 μm. Such particles are better in chemical and physical stability, good material flow characteristics, improving the uniformity of dosage forms and thus suitable for bulk preparation and formulation advantages.

According to another aspect, the present invention provides a process for preparing polymorph Form B of lapatinib ditosylate. Polymorph Form B may be prepared by crystallization from a crystallization solvent containing lapatinib ditosylate. As used herein, the term “crystallization solvent” means a solvent or combination of solvents from which lapatinib ditosylate is preferentially crystallized as polymorph Form B. Representative crystallization solvents for preparation of Form B include water, tetrahydrofuran (THF) and combinations thereof. In a preferred aspect, the crystallization solvent comprises tetrahydrofuran (THF), to which water is gradually added.

In a preferred aspect, Form B of lapatinib ditosylate may be prepared by slurring starting material, crude or pure lapatinib ditosylate, anhydrate or solvate, which can be obtained according to the procedures described in U.S. Pat. No. 7,157,466 (or WO02/02552) with an organic solvent or a mixture of two or more organic solvents under heat. The lapatinib ditosylate is soluble in a mixture of water and THF, but not soluble in non-polar solvent. The preferred organic solvent is tetrahydrofuran. The concentration of lapatinib ditosylate within the solution may range from about 0.1% by weight to the saturation point. This concentration will, of course, vary depending upon the temperature at which the co-solvent solution is held, with warmer temperatures generally allowing for the preparation of more concentrated solutions of lapatinib ditosylate. Preferably, the concentration (w/w %) of lapatinib ditosylate starting material in solution is about 0.5-15%, preferably about 1-10%, more preferably about 1.5-5%. The volume ratio of tetrahydrofuran to water is about 50-95:50-5, preferably about 60-90:40-10, more preferably about 70-85:30-15, most preferably about 75:25. Water is then added into the above suspension, and the mixture is heated, suitably to a temperature in the range of from about 45° C. to 85° C., such as about 50° C. to 75° C., for example about 65-70° C. until all solid materials are dissolved. The clear and hot solution is allowed to cool down to ambient temperature, and the cooled solution is kept at ambient temperature for crystallization. The crystal Form B of lapatinib ditosylate is formed over a period of 1 hour to 24 hours, and the crystal Form B is isolated from the solvent by a vacuum filtration. The isolated crystal From B may be washed with THF, and then dried under a vacuum oven at about 20° C.-65° C., preferably at about 35° C.-55° C., more preferably at about 35° C.-50° C., and most preferably at 42-46° C. for about 5-30 hours to remove the solvent residues.

Once obtained, crystals of polymorph Form B may be used as the nucleating agent or “seed” crystals for subsequent crystallizations of polymorph Form B from the crystallization solvent. In one embodiment, the crystallization solvent is formed by dissolving lapatinib ditosylate in hot tetrahydrofuran and water or other suitable crystallization solvents. The crystallization solvent is then seeded with crystals of polymorph Form B, cooled and filtered, resulting in polymorph Form B. In another embodiment, a crystallization solvent is formed by slurring lapatinib ditosylate in tetrahydrofuran and water or other appropriate solvents. The crystallization solvent is then seeded with crystals of polymorph Form B and filtered, resulting in polymorph Form B. Such seeding with crystals of polymorph Form B may take place at any time during the slurring process. Alternatively, seeding with crystals of polymorph Form B may take place prior to, or simultaneously with, addition of lapatinib ditosylate to the crystallization solvent.

Form B of crystalline lapatinib ditosylate as obtained above is characterized by X-ray powder diffraction pattern, substantially the same as shown in FIG. 2 and Table 2.

Form B of lapatinib ditosylate as obtained above is characterized by differential scanning calorimetry (DSC), substantially the same as shown in FIG. 4.

The crystals of lapatinib ditosylate obtained from recrystallization in solvents as described in above processes may have different crystal habits (e.g., shape), water contents, surface area, bulk or tap density, or particle size, but they clearly still belong to a new and novel polymorphic form (Form B) of lapatinib ditosylate, as it is characterized and confirmed by X-ray powder diffraction pattern and DSC thermogram and TGA. The X-ray powder diffraction pattern of Form B is clearly different from that of other known forms such as lapatinib ditosylate monohydrate, Form A, Form 1, 2, 3, 4, 5 and 6, Form I, II, III, IV, V, VI, VII, VIII, IX, XI, XII, XIII, XIV, XV, XVI, XVII and XVIII or other known forms.

According to a further aspect, the present invention further provides a pharmaceutical composition, which comprises a prophylactically and a therapeutically effective amount of polymorph Form A or Form B of lapatinib ditosylate, substantially as hereinbefore described, together with one or more pharmaceutically acceptable carriers, diluents or excipients, additives, fillers, lubricants, solvents, binders or stabilizers, optionally, one or more other active ingredients.

Pharmaceutical compositions as provided by the present invention can be prepared by known procedures using well-known and readily available ingredients. In preparation of compositions as provided by the present invention, polymorph Form A or Form B of crystalline lapatinib ditosylate, substantially as hereinbefore described, can be mixed with one or more carriers, excipients, diluents, additives, fillers, lubricants, solvents, binders or stabilizers, optionally, one or more other active ingredients.

Pharmaceutical compositions as provided by the present invention can be in the form of tablets, pills, powders, lozenges, sachets, cachets, elixirs, suspensions, emulsions, solutions, syrups, aerosol, ointments soft and hard gelatin capsules, suppositories, sterile injectable solutions and sterile packaged powders containing, for example, up to 70% by weight of polymorph Form A or Form B, substantially as hereinbefore described.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl- and propylhydroxybenzoates, talc, magnesium stearate and mineral oil. The compositions can additionally include lubricating agents, wetting agents, and emulsifying and suspending agents, preserving agents, sweetening agents or flavoring agents.

According to a still another embodiment, the pharmaceutical composition comprises an effective dosage amount of lapatinib ditosylate, wherein lapatinib ditosylate comprises at least a certain percentage of polymorph Form A (based on the total amount of lapatinib ditosylate present in the composition—that is, the total amount of lapatinib ditosylate being 100%). In other words, at least a certain percentage of lapatinib ditosylate present within the pharmaceutical composition exists as polymorph Form A, with the remainder of lapatinib ditosylate being in a different form, including (but not limited to) monohydrate, Form I, II, III, IV, V, VI, VII, VIII, IX, XI, XII, XIII, XIV, XV, XVI, XVII and XVIII, Form 1, 2, 3, 4, 5, and 6 or any other crystalline forms, solvates or amorphous form. More specifically, trace amounts up to 100% Form A, or any amount in between—for example, the active ingredient or drug substance of lapatinib ditosylate in the pharmaceutical composition may comprise less than 0.1%, 0.5%, 1%, 2%, 5%, 10%, 20%, 30%, 40% or 50% by weight of Form A based on the total amount of lapatinib ditosylate in the pharmaceutical composition. Alternatively, the pharmaceutical composition may comprise at least 50%, 60%, 70%, 80%, 90%, 95%, 97%, 98%, 99%, 99.5% or 99.9% by weight of Form A based on the total amount of lapatinib ditosylate in the pharmaceutical composition.

According to a still another embodiment, the pharmaceutical composition comprises an effective dosage amount of lapatinib ditosylate, wherein lapatinib ditosylate comprises at least a certain percentage of polymorph Form B (based on the total amount of lapatinib ditosylate present in the composition—that is, the total amount of lapatinib ditosylate being 100%). In other words, at least a certain percentage of lapatinib ditosylate present within the pharmaceutical composition exists as polymorph Form B, with the remainder of lapatinib ditosylate being in a different form, including (but not limited to) monohydrate, Form I, II, III, IV, V, VI, VII, VIII, IX, XI, XII, XIII, XIV, XV, XVI, XVII and XVIII, Form 1, 2, 3, 4, 5, and 6 or any other crystalline forms, solvates or amorphous form. More specifically, trace amounts up to 100% Form B, or any amount in between—for example, the active ingredient or drug substance of lapatinib ditosylate in the pharmaceutical composition may comprise less than 0.1%, 0.5%, 1%, 2%, 5%, 10%, 20%, 30%, 40% or 50% by weight of Form B based on the total amount of lapatinib ditosylate in the pharmaceutical composition. Alternatively, the pharmaceutical composition may comprise at least 50%, 60%, 70%, 80%, 90%, 95%, 97%, 98%, 99%, 99.5% or 99.9% by weight of Form B based on the total amount of lapatinib ditosylate in the pharmaceutical composition.

The pharmaceutical compositions of the invention may be formulated so as to provide quick, extended, sustained or delayed release of polymorph Form A or Form B of lapatinib ditosylate, substantially as hereinbefore described, after administration to the patient by employing procedures well known in the art. The pharmaceutical compositions of the invention may be preferably formulated so as to provide quick (or immediate), delayed, extended or sustained release tablets consisting of polymorph Form A or Form B of lapatinib ditosylate, substantially as hereinbefore described as active ingredient and plus any additional excipients suitable for preparation of quick, delayed, extended or sustained release tablets.

According to one preferred aspect, the pharmaceutical composition is a quick release formulation. For example, a quick release formulation may comprise lactose or dicalcium phosphate as main diluents, crystalline polymorph Form A or Form B of lapatinib ditosylate as active ingredient, microcrystalline cellulose as a binder or filler, a disintegratant and a lubricant. The dose units are preferably coated with a film coating.

According to one preferred aspect, the pharmaceutical composition is an extended release formulation. For example, an extended release formulation may comprise spheroids comprised of crystalline polymorph Form A or Form B of lapatinib ditosylate, microcrystalline cellulose, and, optionally, hydroxypropylmethylcellulose. The spheroids are preferably coated with a film coating composition comprised of ethyl cellulose and hydroxypropylmethylcellulose.

According to another preferred embodiment, the pharmaceutical composition is a sustained release formulation (e.g., in the form of a tablet). The sustained release formulation may comprise crystalline polymorph Form A or Form B of lapatinib ditosylate, a release rate controlling excipient, and optionally other adjuvants. Suitable rate controlling excipients include, but not limited to, hydroxyalkyl cellulose, such as hydroxypropyl cellulose and hydroxypropyl methyl cellulose (HPMC); poly(ethylene) oxide; alkyl cellulose, such as ethyl cellulose and methyl cellulose; carboxymethyl cellulose; hydrophilic cellulose derivatives; carboxyvinylpolymers (e.g., Carbopol 971P), polyvinylpyrrolidone (PVP) derivatives and polyethylene glycol derivatives.

The sustained release pharmaceutical composition comprises about 1-500 mg of polymorphs Form A or Form B of lapatinib ditosylate and about 15% w/w to about 70% w/w of a release rate controlling pharmaceutical excipients. A preferred sustained release pharmaceutical composition comprises from about 50-300 mg of crystalline polymorphs Form A or Form B of lapatinib ditosylate and about 10% w/w to about 66% w/w of hydroxypropyl methylcellulose, methyl cellulose or ethyl cellulose. Typically, the sustained release formulation provides sustained therapeutically effective plasma levels over at least about 6 or 24-hour period. The peak serum levels during the 6 or 24 hours period are generally up to 5 to 500 ng/mL.

The pharmaceutical compositions are preferably formulated in a unit dosage form, each dosage containing from about 2 to about 800 mg, more usually about 100 to about 500 mg, of polymorph Form A or Form B of lapatinib ditosylate, substantially as hereinbefore described. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier.

A further aspect of the present invention relates to a method of treating or preventing patients suffering from disorders characterized by aberrant erB family PTK activity and certain complications thereof, comprising administering to a patient in need of such treatment an effective amount of a pharmaceutical composition comprising polymorph Form A or Form B of lapatinib ditosylate and a pharmaceutically acceptable carrier.

The present invention further provides polymorph Form A or Form B of lapatinib ditosylate, for use in the manufacture of a medicament for the treatment and/or prophylaxis of patients suffering from disorders characterized by aberrant erB family PTK activity and certain complications thereof.

The particular dose of polymorph Form A or Form B of lapatinib ditosylate, substantially as hereinbefore described, administered according to this invention will of course be determined by the particular circumstances surrounding the case, the route of administration, the particular condition being treated, and similar considerations.

Polymorph Form A or Form B of lapatinib ditosylate, substantially as hereinbefore described, can be administered by a variety of routes including the oral, rectal, transdermal, subcutaneous, intravenous, intramuscular or intranasal routes. A typical daily dose will contain from about 0.01 mg/kg to about 100 mg/kg of polymorph Form A of the present invention. Preferred daily doses will be about 0.01 to about 30 mg/kg, ideally about 1 to about 15 mg/kg.

Having thus described the invention with reference to particular preferred embodiments, those in the art can appreciate modifications to the invention as described and illustrated that do not depart from the spirit and scope of the invention as disclosed in the specification. The following examples are set to illustrate the invention, and aid to understanding the invention, but not intended to, and should not be construed to limit its scope in any way.

### EXPERIMENTAL

Thermogravimetric analysis (TGA) measurements were performed in a Pyris I TGA of Perkin-Elmer (TGA7) under nitrogen purge. The sample was heated from 20° C. to 300° C. at a scan rate of 10° C./minute.

DSC measurements were performed in a TA instrument with a sealed pan at a scan rate of 10° C./minute from 40° C. to 300° C. under nitrogen purge.

X-ray powder diffraction (X-RPD) data were obtained by ARL X-Ray powder diffractometer model XTRA-030. Scanning range 3-50 deg. 2 theta, continuous scan, rate 3 deg./min. The accuracy of peak positions was defined as +/−0.2 degrees due to such experimental differences as instrumentation and sample preparation etc.

### EXAMPLES

### Example 1

### Preparation of Polymorph Form A of Lapatinib Ditosylate

Lapatinib ditosylate (2.2 g) was suspended in about 35 ml boiling tetrahydrofuran (THF, HPLC grade). To the suspension was added about 5-6 ml boiling water and the suspension was heated up and stirred until all solid materials are dissolved. The resulting clear solution was then cooled down to ambient temperature and then kept at ambient temperature for overnight for recrystallization. Next morning, nice crystals were formed. The recrystallization at ambient temperature continued for two more days. The resulting crystals were isolated by filtration and dried in vacuum oven at about 40° C. for 15 hours and then at about 25-30° C. for 12 hours to give a yellowish crystalline solid (about 1.7 g). DSC, FT-IR, TGA and X-ray diffraction pattern techniques were used to characterize the obtained product. DSC of the obtained product showed an exothermic peak at about 139° C. and an endothermic peak at about 253° C., as shown in FIG. 3. Powder X-ray diffraction pattern of the obtained product is shown in FIG. 1. The TGA, as shown in FIG. 5, indicated that the obtained product contains about 9% (w/w) water. The obtained product is a hydrate form (Form A) of lapatinib ditosylate.

### Example 2

### Preparation of Polymorph Form B of Lapatinib Ditosylate

Lapatinib ditosylate (2.0 g) was suspended in about 30 ml near boiling tetrahydrofuran (THF, HPLC grade). To the suspension was added about 5-6 ml near boiling water. The suspension was heated up and stirred until all solid materials were dissolved. About 300 mg lapatinib ditosylate was added into the clear solution under heating, and continued stirring until the solution turned into clear. The resulting clear solution was then cooled down to ambient temperature by air and then kept at ambient temperature for recrystallization. After 2-4 hrs, lots of crystals were formed. The recrystallization continued overnight. Next morning, the resulting crystals were isolated by vacuum filtration. The isolated crystals were washed with THF and dried in vacuum oven at about 45° C. for 7 hours to give a yellowish crystalline solid (about 1.6 g). DSC, TGA and X-ray diffraction pattern techniques were used to characterize the obtained product. DSC of the obtained product showed three endothermic peaks at about 144° C. and 252° C., respectively, as shown in FIG. 4. Powder X-ray diffraction pattern of the obtained product is shown in FIG. 2. The TGA of the obtained product indicated that the obtained product contains less than 0.2% (w/w) water, as shown in FIG. 8. The obtained product is a new form of lapatinib ditosylate (Form B).

### Example 3

### Preparation of Amorphous Lapatinib Ditosylate

Method A: Lapatinib ditosylate (1.5 g) was completely dissolved in a mixture of water (50 ml) and methanol (50 mL) in a round bottom flask under heating at 40-50° C. to obtain a clear solution. The solution was then transferred to a heavy walled flask (2 liters). The solution in flask is rapidly cooled by liquid nitrogen until it becomes a frozen solid material. The frozen solid material in the flask was evacuated and maintained under about 0.01 mm Hg vacuum for about 36 hours. The product was further dried under vacuum at 30-40° C. for 24 hours to afford 1.2 g of the desired product of amorphous lapatinib ditosylate. The sample was submitted for powder X-ray analysis, which confirmed that the resulting substance was in amorphous form.

Method B: Lapatinib ditosylate (6 g) was dissolved in 600 ml of water and ethanol (1:1, v/v), and the suspension mixture is heated to 40-50° C. to obtain a clear solution. The hot solution was cooled to ambient temperature (25-30° C.), and then subjected to spray solvent removing procedure in a Mini-Spray Dryer (e.g., Buchi Model-190) at an inlet temperature 120-150° C. and outlet temperature 70-90° C. using nitrogen gas. A light-white powder of lapatinib ditosylate in an amorphous form was obtained. The product was further dried under vacuum at 30-40° C. for 24 hours to afford 5.1 g of the desired product of amorphous lapatinib ditosylate. The powder X-ray diffractogram showed that the resulting substance was in amorphous form.

### Example 4

### Preparation of Solid Amorphous Dispersion of Lapatinib Ditosylate and PVP

Method A: Lapatinib ditosylate (2.0 g) and polyvinylpyrrolidone (PVP, K=30) (4.0 g) was dissolved in 200 ml of water and ethanol (1:1, v/v), and the suspension mixture is heated to 40-50° C. to obtain a clear solution. The hot solution was cooled to ambient temperature (25-30° C.), and then subjected to spray solvent removing process in a Mini-Spray Dryer (e.g., Buchi Model-190) at an inlet temperature 119-148° C. and outlet temperature 75-85° C. using nitrogen gas. A light-yellow powder of solid dispersion containing lapatinib ditosylate and PVP in an amorphous form was obtained. The product was further dried under vacuum at 40° C. for 36 hours to afford 5.0 g of the desired solid amorphous dispersion of lapatinib ditosylate and PVP. The powder X-ray diffractogram showed that the resulting substance was in amorphous form.

Method B: 2.0 g lapatinib ditosylate and 4.0 g PVP-VA64 (Plastone S-630, K=26-34) was dissolved in ethanol (200 ml) at ambient temperature, and suspension mixture was heated to 50° C. to obtain a clear solution. The solvent was evaporated through a distillation process under vacuum (30-80 mm Hg) at about 40° C. to about 70° C. or under reflux. The product was then isolated (about 5.5 g) when no visible liquid was remained and the drying was continued under vacuum at about 40° C. for 24-48 hours to remove the solvent. The powder X-ray diffractograrm and DSC of the solid material showed that the resulting substance was amorphous product.

Method C: 2.0 g lapatinib ditosylate and 4.0 g hydroxylpropylmethyl cellulose (HPMC) was dissolved in ethanol (200 ml) at ambient temperature, and suspension mixture was heated to 50° C. to obtain a clear solution. The solvent was evaporated through a distillation process under vacuum (30-80 mm Hg) at about 40° C. to about 70° C. or under reflux. The product was then isolated (about 5.1 g) when no visible liquid was remained and the drying was continued under vacuum at about 40° C. for 48 hours to remove the solvent. The powder X-ray diffractograrm and DSC of the solid material showed that the resulting substance was amorphous product.

### Example 5

### Formulation of Tablets Containing Crystalline Form A or Form B of Lapatinib Ditosylate

There were three major steps involved in manufacturing the tablets: (A) preparation of polymorphic Form A or Form B of lapatinib ditosylate granular concentrate; (B) preparation of tablet core; (C) coating the tablet core. The amount of each ingredient included in the formulation is shown in Table 2 and Table 3 (quantity in gram).

### A: Preparation of Polymorph Form A or Form B of Lapatinib Ditosylate Granular Concentrate

The following ingredients (quantity in gram) were sifted through a clean screen (typically 0.066″): lactose anhydrous, dicalcium phosphate anhydrous, pregelatinized starch, sodium starch glycolate and microcrystalline cellulose.

The screened materials were transferred into a high shear (high-energy) mixer and blended for ten (10) minutes at 100 rpm. The blended material was granulated with purified water. The wet granules were passed through a screen (typically 0.132″), and dried in a fluid bed drier until loss on drying is less than 0.2-0.5% w/w.

TABLE 3
% Composition of Form A or Form B of Lapatinib Ditosylate (52%,
w/w, calculated as lapatinib free base) Granular Concentrate

| Granular concentrate batch # | 1 | 2 |
|---|---|---|
| Form A or Form B of lapatinib | 328 | 328 |
| ditosylate | | |
| Lactose anhydrous | 50 | |
| Dicalcium phosphate anhydrous | | 50 |
| Sodium starch glycolate | 10 | 10 |
| Pregelatinized starch | 10 | 20 |
| Microcrystalline cellulose | 30 | 20 |
| Purified water* | | |

*Water was removed during the process

The dried granules were passed a screen (typically 0.039″) and blended using a tumble blender for 10 minutes at 12 rpm.

### B: Preparation of Tablet Core Comprising Form A or Form B of Lapatinib Ditosylate

The concentrated granules are placed into a tumble blender. About two thirds of the lactose or dicalcium phosphate is screened and added to the blender, and blended for ten (10) minutes. The microcrystalline cellulose, sodium starch glycollate, magnesium stearate and remaining lactose or dicalcium phosphate are screened and added to the blender. The mixtures are blended together for ten (10) minutes. The blended material was compressed on a Kikusui Libra tablet compression machine to a target weight of 600 mg for the 200 mg lapatinib tablets, and to a target weight of 700 for 250 mg lapatinib tablets.

### C: Preparation of Coated Tablet Comprising Form A or Form B of Lapatinib Ditosylate

The tablet cores are then transferred to a tablet-coating machine (pan coater). The tablet bed was pre-heated with warm air (approximately 60° C.). The pan speed

TABLE 4
% Composition of Tablet Core and Coated
Tablets (quantity, mg per tablet)

Formulation batch#

| | |
|---|---|
| 1 | 2 |

Dosage strength (calculated
from lapatinib free base)

| | 200 mg | 250 mg |
|---|---|---|
| Form A or Form B of lapatinib | 384 | 480 |
| ditosylate concentrate granules | | |
| Microcrystalline cellulose | 211 | 215 |
| Magnesium stearate | 5 | 5 |
| Total weight | 600 | 700 |
| Coating material | 10.0 | 15.0 |
| Total weight of coated tablet | 610.0 | 715.0 |

was adjusted to 5-9 RPM before starting the spray cycle. The spray cycle was activated. The exhaust temperature was maintained between 40° C. and 50° C. throughout the cycle. After the proper amount of solution was applied, the coated tablets were dried for approximately two (2) minutes. Steps were repeated for all pans to coat all tablets in the batch and film coated until the tablet weight has increased by 1.0% to 3.5%. All tablets were packaged in plastic bottles with desiccants, and the bottles were heat sealed, then placed under the stress condition.

### Example 6

### Stability Studies

The stability of Form A or Form B of lapatinib ditosylate bulk material and tablets is assessed by storing samples for up to 12 weeks at 25° C./60% RH or up to 5 weeks at 40° C./75% RH. Changes are monitored using a stability-indicating HPLC method. Results were calculated by normalized peak area (npa). Degradants are identified by comparison of their relative retention times against impurity standards.

### (i) Polymorph Form A or Form B of Lapatinib Ditosylate Bulk Material

Polymorph Form A or Form B of lapatinib ditosylate bulk material was stable with respect to polymorphic form (or phase) stability as well as formation of known and unknown degradants for over 3 months when stored under normal conditions of temperature and humidity. Similarly, polymorph (phase) and chemical stability of Form A was demonstrated at elevated temperatures and humidity (40° C./75%) for over 5 weeks.

### ii) Tablets Comprising Polymorph Form A or Form B of Lapatinib Ditosylate

Tablets comprising polymorph Form A or Form B of lapatinib ditosylate was stable with respect to the formation of known and unknown degradants for over 6 months when stored under normal manufacturing and storage conditions of temperature and humidity (25° C./65% relative humidity).

We claim:

1. Polymorph Form A of lapatinib ditosylate.

2. The Form A of claim 1, characterized as having an X-ray diffraction pattern with characteristic peaks (expressed in 2θ±0.2° 2θ) at one or more of the following positions: 3.86, 4.80, 14.2, 15.96, 19.42 or 19.88.

3. The Form A of claim 1, characterized as having X-ray powder diffraction pattern substantially the same as that shown in FIG. 1.

4. The Form A of claim 1, wherein the Form A is the hydrate lapatinib ditosyalte and the molecular molar ratio of lapatinib:tosylate:water in Form A being about 1:2:2-7.

5. Polymorph Form B of lapatinib ditosylate.

6. The Form B of claim 5, characterized as having an X-ray diffraction pattern with characteristic peaks (expressed in 2θ±0.2° 2θ) at one or more of the following positions: 5.10, 9.00, 15.57, 19.10, 19.85, 21.20, 22.20, 27.00 or 28.25.

7. The Form B of claim 5, characterized as having X-ray powder diffraction pattern substantially the same as that shown in FIG. 2.

8. The composition of claim 5, wherein the composition comprising less than 0.1% to at least 99.9% by weight of polymorph Form B based on the total weight of lapatinib ditosylate in the composition.

9. The composition of claim 5, wherein the composition comprising less than 2% by weight of polymorph Form B based on the total weight of lapatinib ditosylate in the composition.

10. The composition of claim 5, wherein the composition comprising at least 50% by weight of polymorph Form B based on the total weight of lapatinib ditosylate in the composition.

11. The composition of claim 5, wherein the composition comprising at least 95% by weight of polymorph Form B based on the total weight of lapatinib ditosylate in the composition.

12. The composition of claim 5, wherein the composition comprising at least 99.9% by weight of polymorph Form B based on the total weight of lapatinib ditosylate in the composition.

13. A pharmaceutical composition comprising polymorph Form A, Form B, amorphous form or amorphous solid dispersion of lapatinib ditosylate with one or more pharmaceutically acceptable carriers, excipients, diluents, additives, fillers, lubricants or binders.

14. The pharmaceutical composition of claim 13, wherein lapatinib ditosylate comprising less than 0.1% to at least 99.9% by weight of polymorph Form A based on the total weight of lapatinib ditosylate in the pharmaceutical composition.

15. The pharmaceutical composition of claim 13, wherein lapatinib ditosylate comprising less than 0.1% to at least 99.9% by weight of polymorph Form B based on the total weight of lapatinib ditosylate in the pharmaceutical composition.

16. The pharmaceutical composition of claim 13, wherein the composition comprising amorphous solid dispersion of lapatinib ditosylate with one or more pharmaceutically acceptable polymer.

17. The pharmaceutical composition of claim 13 or 16, wherein lapatinib ditosylate comprising less than 2% to at least 99.9% by weight of amorphous lapatinib ditosylate based on the total weight of lapatinib ditosylate in the pharmaceutical composition.

18. The pharmaceutical composition of claim 17, wherein the polymer is selected from a group consisting of hydroxy-propylcellulose (HPC), hydroxypropylmethylcellulose (HPMC), hydroxypropyl-methylcellulose acetate succinate (HPMC-AS), polyvinylpyrrolidone (PVP) and co-polymers thereof with PVP, cross-linked PVP, PVP-VA64, polyethyleneglycol 8000 and polyethyleneglycol 6000, polyethylene-/polypropylene-/polyethylene-oxide block copolymer, pluronic F68, Eudragit®L-1 00-55 and Eudragit®E-100, α-cyclodextrin, β-cyclodextrin and its derivatives, hydroxylpropyl-β-cyclodextrin and its derivatives.
